(19) United States
(12) Patent Application Publication
(10) Pub. No.: US 2003/0129250 A1
(43) Pub. Date: Jul. 10, 2003

(54) Particulate compositions for improving solubility of poorly soluble agents

(75) Inventors: Richard P. Batycky, Newton, MA (US); George Grandolfi, Milford, OH (US); Sean Plunkett, Westborough, MA (US); Michael M. Lipp, Framingham, MA (US); James Wright, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Advanced Inhalation Research Inc., Cambridge, MA (US)

(21) Appl. No.: 10/300,726

(22) Filed: Nov. 20, 2002

Related U.S. Application Data
(60) Provisional application No. 60/331,810, filed on Nov. 20, 2001.

(51) Int. Cl. A61K 31/58; A61K 9/14; A61K 9/16

(52) U.S. Cl. 424/490; 514/176

(57) ABSTRACT

The invention is drawn to particles for oral drug delivery produced by spray-drying a dilute solution of a poorly soluble agent. The particles comprise regions of poorly soluble agent wherein the dissolution rate enhancement is between about 2-fold and about 25-fold compared to the agent in bulk form.

### RELATED APPLICATION

This application claims the benefit of U.S. Provisional Application No. 60/331,810, filed Nov. 20, 2001.

### BACKGROUND OF THE INVENTION

The solubility of a drug can have a significant effect on bioavailability regarding the rate and extent of oral absorption. Low solubility profoundly reduces the rate of dissolution of the drug, and thus reduces the rate and extent of intestinal uptake of the drug.

Technologies developed to address poor solubility of drugs include particle treatment (e.g., complexation and dispersion) and particle modification (e.g., coating, particle size reduction and precipitation).

An example of particle treatment is dispersion, involving the formation of drug-containing liposomes, vesicles, emulsions or colloidal suspensions. Dispersions are limited by the stability of the drug, variability upon mixing and the need for organic solvents. Dispersions for oral administration also typically have a poor taste.

A first example of particle modification is particle coating with a dispersive agent. This process is limited most significantly by the particle's melting point because the coating process requires elevated temperatures that can cause excessive agglomeration and annealing. A melting point of greater than 70° C. is recommended. Other limitations include cost, complexity, and potential yield loss because the process adds a second step.

A second example of particle modification is particle size reduction, including for example, attrition or milling. Problems associated with these high energy forms of reduction are unwanted polymorphic transition, agglomeration, poor flowability and poor wettability. Techniques for making micron and sub-micron sized particles are very energy intensive and thus may lead to degradation of the drug.

Attrition, or size reduction through application of force parallel to the particle surface (e.g. shear-induced forces), shares some of the shortcomings of milling. Physical force is applied to the particles, usually in a carrier solvent. The transport of particles at high flow rates through a high pressure drop to induce shear can heat the solvent, causing degradation of the active material. Multiple passes are often required, exacerbating this degradation. After attrition, the particles must be recovered from the solvent phase necessitating a time-consuming filtration and drying process.

Milling (e.g., grinding, rolling or impaction) requires stable solids. This is problematic for proteins, polypeptides and some larger organic molecules as these species may be shear-sensitive and degrade during milling. These methods can also contribute particles of foreign material (e.g., fragments of the apparatus) to the drug.

A third example of particle modification is water-mediated precipitation from an organic solvent. This process requires the removal of organic solvent from the aqueous suspension to achieve acceptable residual levels by isolating, rinsing and re-suspending the particles. This is a laborious and costly process step, especially if the solvent is toxic. The material is then either left in suspension (possibly with a surfactant) or lyophilized to produce a dry powder. If a substance cannot be stored as a suspension, it must be lyophilized, which is an undesirable process step. Lyophilization requires costly capital equipment, represents an additional process step and generally has a slow throughput (on the order of days/batch) compared to other unit operations.

### SUMMARY OF THE INVENTION

Applicants have discovered an improved way to achieve dissolution of poorly soluble drugs without sacrificing targeted flowability, wettability, selective agglomeration or annealing, yield or polymorphic stability. The known methods to address poor solubility of drugs, as described herein, have significant limitations and thus have not found wide-ranging application. As the present discovery shows, it is desirable to dissolve poorly soluble drugs in a safe cost effective manner, to overcome limitations such as, but not limited to, degradation, introduction of impurities, loss of selective agglomeration or annealing, loss of yield, and/or toxicity.

One method to achieve dissolution of poorly soluble drugs is to increase the surface area in contact with the solvent. This is accomplished by creating particles, comprising one or more drugs, which are relatively large (significantly larger than a micron) yet comprised of very thin walls (several hundred nanometers or less) and having high surface area. Such particles exhibit the rapid dissolution typically associated with very small nanoparticles, yet still have the excellent flowability and dispersibility of large porous particles. The particles comprise submicron drug nanoparticles embedded in the particle walls and/or drug molecularly dispersed with one or more excipients. Alternatively, the particles comprise 100% drug. Compared to the methods described above (e.g., milling or precipitation), the formation of large porous particles by spray drying is a mild process that leads to a stable product.

The particles of the invention are produced by spray-drying a dilute solution (e.g, less than about 5%, 3% or about 1% (w/v), immediately prior to spray drying) of a therapeutic, prophylactic, or diagnostic agent (hereinafter collectively referred to as “drug” or “agent”) which is poorly soluble. In one embodiment, the particles comprise one or more excipients such as, for example, biodegradable excipients.

One embodiment of the invention involves a particulate composition for drug delivery comprising biodegradable particles incorporating a therapeutic, prophylactic or diagnostic agent and optionally containing one or more excipients. The particles have a tap density less than about 0.4 g/cm³ and a mean geometric diameter of at least about 5 microns, for example, about 5 microns to about 30 microns. The thickness of the particle walls can be about 50 nanometers to about 400 nanometers. The particles are effective to yield a dissolution rate enhancement of the particles of at least about 2-fold over the bulk crystalline drug. In one embodiment, the particles are effective to yield a dissolution rate enhancement of the particles of at least about 10-fold over the crystalline drug in bulk form. Preferably, the particles are effective to yield a dissolution rate enhancement of about 2-fold to about 25-fold over the crystalline drug in bulk form, such as a dissolution rate enhancement of about 2-fold to about 10-fold or about 10-fold to about 25-fold over the bulk crystalline drug.

Practice of the present invention increases dissolution for two main reasons. The first reason is that particles exhibit an exponential increase in solubility as their size falls below 1 micron. Particles of the present invention may comprise one or more drugs wherein one or more drugs form 100% of the particles' composition, wherein a drug forms a solid solution with one or more excipients such as, for example, where a drug is molecularly dispersed with one or more excipients and/or wherein, a drug is present in regions of drug-rich material such as, for example, where a drug is present in drug nanoparticles. The regions of drug in the particle are of very small size such as about 50 nanometers to about 500 nanometers, for example, about of about 200 nanometers to about 500 nanometers, and may be larger than the particle wall. As the excipients of the particle dissolve, the regions of the drug act as separate particles thereby increasing solubility. The drug, whether in a solid solution or a drug rich region, has a diffusion/dissolution path length in the solid state of half of the wall thickness. A second reason is that amorphous particles dissolve more quickly than the same bulk compound in crystalline form. Drug domains formed by spray drying are either amorphous or crystalline.

The increase in dissolution rate permits a decrease of the necessary dosage of a drug or more effective delivery of drugs that were previously only sparingly soluble.

### BRIEF DESCRIPTION OF THE DRAWINGS

FIG. 1 is a graph showing the dissolution rate enhancement of danazol. The graph shows measurements of dissolution taken at intervals over a time of 15 min. Dissolution is indicated as percent dissolved. Time is indicated in minutes. Formulation particles are indicated as “♦”. Bulk agent particles are indicated as “▪”.

FIG. 2 is a graph showing the dissolution rate enhancement of glyburide. The graph shows measurements of dissolution taken at intervals over a time of 15 min. Dissolution is indicated as percent dissolved. Time is indicated in minutes. Formulation particles are indicated as “♦”. Bulk agent particles are indicated as “▪”.

FIG. 3 is a graph showing the dissolution rate enhancement of glipizide. The graph shows measurements of dissolution taken at intervals over a time of 15 min. Dissolution is indicated as percent dissolved. Time is indicated in minutes. Formulation particles are indicated as “♦”. Bulk agent particles are indicated as “▪”.

FIG. 4 is a graph showing the dissolution rate enhancement of lansoprazole. The graph shows measurements of dissolution taken at intervals over a time of 15 min. Dissolution is indicated as percent dissolved. Time is indicated in minutes. Formulation particles are indicated as “♦”. Bulk agent particles are indicated as “▪”.

FIG. 5 is a graph showing the dissolution rate enhancement of piroxicam. The graph shows measurements of dissolution taken at intervals over a time of 15 min. Dissolution is indicated as percent dissolved. Time is indicated in minutes. Formulation particles are indicated as “♦”. Bulk agent particles are indicated as “▪”.

FIG. 6 is a graph showing the dissolution rate enhancement of ketoprofen. The graph shows measurements of dissolution taken at intervals over a time of 15 min. Dissolution is indicated as percent dissolved. Time is indicated in minutes. Formulation particles are indicated as “♦”. Bulk agent particles are indicated as “▪”.

FIG. 7 is a schematic drawing showing one embodiment of the poorly soluble agent embedded in the walls of the particle. Particle walls are represented as “ [symbol] ”. Poorly soluble agent is represented as “ [symbol] ”.

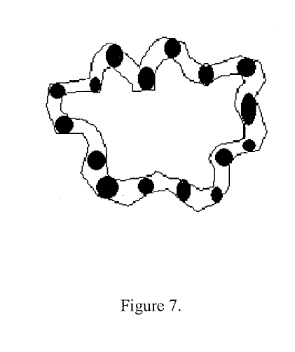

FIG. 8 is a schematic drawing representing an alternative embodiment of the poorly soluble agent molecularly dispersed within the walls of the particle, that is, the agent is not present as drug nanoparticles. Particle walls are represented as “

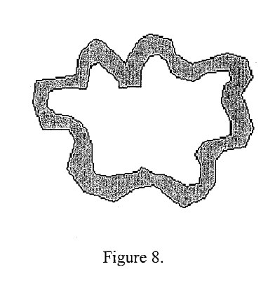

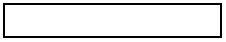

”.

FIG. 9 is a graph showing blood serum concentration of glyburide, in nanograms per milliliter, versus time, in hours, following oral dosing of four groups of Sprague-Dawley rats using (a) a mixture of glyburide, maltodextrin, leucine, and DPPC (‘Bulk’); (b) spray dried powder comprising glyburide, maltodextrin, leucine, and DPPC (‘Spray Dried’); (c) non-micronized glyburide (‘Standard’); and (d) micronized glyburide (‘Micronized’).

### DETAILED DESCRIPTION OF THE INVENTION

This invention concerns inclusion of poorly-soluble compounds into porous amorphous, low density particles. The particles are produced by spray drying a dilute solution (e.g., less than about 10 g/L) comprising a poorly soluble compound and any desired excipients. The droplets are spray dried, creating porous particles which are collected at the chamber outlet. The dilute solution is formulated using solvents such as, but not limited to, methanol; ethanol; n-propanol; isopropanol; n-butanol; 2-butanol; isobutanol; volatile ketones such as acetone, 2-butanone; volatile esters such as ethyl acetate, propyl acetate, 1,4-dioxane, tetrahydrofuran, and diethyl ether; and excipients to produce the final powder such as, but not limited to, surfactants, matrix builders and stabilizers. In one embodiment, the solvent has some water miscibility.

While not intending to be bound to one particular theory, the particles that comprise the final powder have a short diffusion path length due to wall thickness. A “short diffusion path length” refers to about half the wall thickness of a particle. Preferably, the particles are significantly larger than one micron. For example, in one aspect, the particles have a mean size (e.g., a mean geometric diameter) of at least about 5 microns.

In one aspect, the size distribution of a sample of particles is substantially symmetric with respect to the median aerodynamic diameter (e.g., the mass median aerodynamic diameter). As those skilled in the art recognize, a sample of particles will have a median aerodynamic diameter and a mean aerodynamic diameter (e.g., a mass median aerodynamic diameter and a mass mean aerodynamic diameter) that are substantially equal when the particle size distribution is substantially symmetric with respect to the median aerodynamic diameter (e.g., the mass median aerodynamic diameter). Likewise, a sample of particles will have a median geometric diameter and a mean geometric diameter (e.g., a volumetric median geometric diameter and a volumetric mean geometric diameter) that are substantially equal when the particle size distribution is substantially symmetric with respect to the median geometric diameter (e.g., the volumetric median geometric diameter). Alternatively, the size distribution of a sample of particles is not substantially symmetric with respect to the median aerodynamic diameter (e.g., the mass median aerodynamic diameter) or the median geometric diameter (e.g., the volumetric median geometric diameter). In such a case, one skilled in the art recognizes that the mean aerodynamic diameter (e.g., the mass mean aerodynamic diameter) and/or the mean geometric diameter (e.g., the volumetric mean geometric diameter) differ from the median aerodynamic diameter and/or the median geometric diameter, respectively. In this instance, the particles are further characterized by the mean aerodynamic diameter and/or the mean geometric diameter of the sample of particles. One skilled in the art can calculate one given the other.

In another embodiment, the particles have a mean size (e.g., a mean geometric diameter) of about 5 to about 50 microns, about 5 to about 30 microns, about 5 to about 25 microns, or about 5 to about 15 microns. In one embodiment, the particles have a tap density of less than about 0.4 g/cm³ such as tap densities of less than about 0.3, 0.2, 0.1, 0.05, or less than about 0.01 g/cm³. Preferably, the particles have a mean size of (e.g., a mean geometric diameter) about 5 microns to about 25 microns and have a tap density of less than about 0.2 g/cm³ such as, for example, about 0.01 to about 0.2 g/cm³. More preferably, the particles range in size from about 5 to about 15 microns (e.g., about 5 to about 12 microns) and have a tap density of about 0.01 to about 0.1 g/cm³. The particles' surface area (e.g., the particles' average surface area) is typically about 1 m²/g to about 50 m²/g, but, under certain circumstances, may be larger or smaller than this range. Preferably, the particles' surface area is about 2 m²/g to about 40 m², about 5 m²/g to about 30 m², or about 5 m²/g to about 25 m²/g. In one aspect, the invention is directed to particles having wall thicknesses of about 50 nanometers to about 500 nanometers, about 50 nanometers to about 400 nanometers, about 100 nanometers to about 300 nanometers, or about 150 nanometers to about 250 nanometers, preferably about 200 nanometers. The particles are effective to yield a dissolution rate enhancement of the particles of about 2-fold to about 25-fold compared to the agent in bulk form, more preferably a dissolution rate enhancement of particles of about 2-fold to about 10-fold. The regions of drug in the particle are small in size such as about 50 to about 500 nanometers, e.g., about 200 to about 500 nanometers. In other embodiments, regions of drug in the particle are about 50 to about 400 nanometers, about 100 nanometers to about 300 nanometers, or about 150 nanometers to about 250 nanometers and are preferably less than or equal to about 200 nanometers. The incorporation of poorly soluble drugs does not interfere with the unique features of the low density particle.

In one embodiment, the larger size and the more highly convoluted morphology of the particles contribute to make them easily dispersible and stable with respect to agglomeration during storage. Particles with diameters of less than about 5 microns are prone to sticking together or agglomerating, with this tendency increasing as diameter decreases. The morphology of the instant particles contributes to enhanced dispersibility and stability by decreasing the area of contact between particles. The surface contact is minimized by presence of numerous folds and convolutions. The radially-exposed surface is thus reduced as the particle surface is dominated by crevices which cannot interact chemically during contact with other particles.

In one embodiment of the invention, the particles deliver at least about 5 mg of the drug. In other embodiments, the particles deliver at least about 10, 50, 100, or about 200 mg of drug. Higher amounts can also be delivered, for example, the particles can deliver at least about 100 mg of agent. The powder can be compressed about 10 to about 29 times. Even when compressed, particles of the instant invention still retain the improvement in dissolution rate. Typical compressed tablets comprise as much as 1000 mg of a drug. Alternatively, a capsule with a volume of about 0.5 cm³ can be filled with dry powder particles to create a dose of about 250 mg and thus a typical regimen of 2 capsules would provide a dose of about 500 mg.

In another embodiment of the invention the particles include a surfactant. As used herein, the term “surfactant” refers to any agent which preferentially adsorbs to an interface between two immiscible phases, such as the interface between water and an organic polymer solution, a water/air interface or organic solvent/air interface. Surfactants generally possess a hydrophilic moiety and a lipophilic moiety, such that, upon absorbing to microparticles, they tend to present moieties to the external environment that do not attract similarly-coated particles, thus reducing particle agglomeration. Surfactants may also promote absorption of a therapeutic or diagnostic agent and increase bioavailability of the agent.

Suitable surfactants which can be employed in fabricating the particles of the invention include but are not limited to hexadecanol; fatty alcohols such as polyethylene glycol (PEG); polyoxyethylene-9-lauryl ether; surface active fatty acids, such as palmitic acid or oleic acid; glycocholate; surfactin; poloxamers; sorbitan fatty acid esters such as sorbitan trioleate (e.g., Span® 85, Span® is a trademark of ICI Americas, Inc.); polyoxyethylene sorbitan fatty acid esters such as polyoxyethylene 20 sorbitan monooleate (e.g., Tween® 80, Tween® is a trademark of ICI Americas, Inc.) and tyloxapol. In one embodiment, the surfactant can be present in the particles in an amount ranging from about 0 to about 90 weight percent. In another embodiment, the surfactant can be present in the particles in an amount of about 5 to about 80, about 5 to about 70, about 10 to about 60, about 10 to about 50, about 10 to about 40, or about 10 to about 30 weight percent, such as about 10 to about 20, or about 10 weight percent.

Methods of preparing and administering particles including surfactants, and in particular phospholipids, are disclosed in U.S. Reissue Pat. No. RE 37,053 to Hanes, et al., (formerly U.S. Pat. No. 5,855,913, issued on Jan. 5, 1999) and in U.S. Pat. No. 5,985,309, issued on Nov. 16, 1999 to Edwards, et al., The teachings of both are incorporated herein by reference in their entirety.

In a further embodiment, the particles include other excipients such as, for example buffer salts, dextran, polysaccharides, lactose, trehalose, cyclodextrins, proteins, polycationic complexing agents, peptides, polypeptides, fatty acids, fatty acid esters, inorganic compounds, phosphates, lipids, sphingolipids, cholesterol, surfactants, polyaminoacids, polysaccharides, proteins, salts, gelatins, and polyvinylpyrridolone, among others.

Celluloses include, for example, microcrystaline cellulose (Avicel®, FBC Biopolymer, Philadelphia, Pa.), HPMC, and carboxymethylcellulose. Gums include, for example, gum tragacanth, and acacia. Starches include, for example, sodium starch glycollate, modified corn starch, and pregelatinized starch. Inorganic salts include for example, calcium phosphate dihydrate and sodium chloride. Dextrins include, for example, maltodextrins and cyclodextrins. Saccharides include, for example, lactose, trehalose, sucrose, and dextrose.

Typical tableting excipients can be used such as diluents, binders and disintegrants. Suitable diluents include, for example, lactose dextrose, mannitol, starches and dicalcium phosphate. Suitable binders include, for example, gums, starches and celluloses. Suitable disintegrants include, for example, alginic acid and sodium alginate.

It is understood, however, that in certain embodiments, the particles are substantially free of polycationic complexing agents, in particular, protamine.

In yet another embodiment of the invention, the particles also include one or more amino acids. Suitable amino acids include naturally occurring and non-naturally occurring hydrophobic and hydrophilic amino acids. Some suitable naturally occurring hydrophilic amino acids include, but are not limited to, glycine, aspartic acid and glutamic acid. Some suitable naturally occurring hydrophobic amino acids include, but are not limited to, leucine, isoleucine, alanine, valine, phenylalanine, glycine and tryptophan. Combinations of amino acids can also be employed. Furthermore, combinations of hydrophobic and hydrophilic (preferentially partitioning in water) amino acids, where the overall combination is hydrophobic, can also be employed. Combinations of one or more amino acids and one or more phospholipids or surfactants can also be employed. Non-naturally occurring amino acids include, for example, beta-amino acids. Both D, L configurations and racemic mixtures of hydrophobic amino acids can be employed. Suitable hydrophobic amino acids can also include amino acid derivatives or analogs. As used herein, an amino acid analog includes the D or L configuration of an amino acid having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. As used herein, aliphatic groups include straight chained, branched or cyclic C1-C8 hydrocarbons which are completely saturated, which contain one or two heteroatoms such as nitrogen, oxygen or sulfur and/or which contain one or more units of unsaturation. Aromatic groups include carbocyclic aromatic groups such as phenyl and naphthyl and heterocyclic aromatic groups such as imidazolyl, indolyl, thienyl, furanyl, pyridyl, pyranyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F) —O (aliphatic, substituted aliphatic, benzyl, substituted benzyl, aryl or substituted aryl group), —CN, —NO₂, —COOH, —NH₂, —NH (aliphatic group, substituted aliphatic, benzyl, substituted benzyl, aryl or substituted aryl group), —N (aliphatic group, substituted aliphatic, benzyl, substituted benzyl, aryl or substituted aryl group)₂, —COO (aliphatic group, substituted aliphatic, benzyl, substituted benzyl, aryl or substituted aryl group), —CONH₂, —CONH (aliphatic, substituted aliphatic group, benzyl, substituted benzyl, aryl or substituted aryl group)), —SH, —S (aliphatic, substituted aliphatic, benzyl, substituted benzyl, aromatic or substituted aromatic group) and —NH—C(═NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more substituents. Modifying an amino acid substituent can increase, for example, the lypophilicity or hydrophobicity of natural amino acids which are hydrophilic.

A number of the suitable amino acids, amino acids analogs and salts thereof can be obtained commercially. Others can be synthesized by methods known in the art. Synthetic techniques are described, for example, in Greene and Wuts, “Protecting Groups in Organic Synthesis,” John Wiley and Sons, Chapters 5 and 7, 1991.

Hydrophobicity is generally defined with respect to the partition of an amino acid between a nonpolar solvent and water. Hydrophobic amino acids are those acids which show a preference for the nonpolar solvent. Relative hydrophobicity of amino acids can be expressed on a hydrophobicity scale on which glycine has the value 0.5. On such a scale, amino acids which have a preference for water have values below 0.5 and those that have a preference for nonpolar solvents have a value above 0.5. As used herein, the term “hydrophobic amino acid” refers to an amino acid that, on the hydrophobicity scale, has a value greater or equal to 0.5, or in other words, has a tendency to partition in the nonpolar acid which is at least equal to that of glycine.

The amino acid can be present in the particles of the invention in an amount from about 0 weight percent to about 60 weight percent. Preferably, the amino acid can be present in the particles in an amount of about 5 to about 50 weight percent, about 5 to about 40 weight percent, about 5 weight percent to about 30 weight percent, for example, the amino acid can be present in an amount of about 20 to about 40 weight percent, about 25 to about 35 weight percent, or about 30 weight percent. The salt of a hydrophobic amino acid can be present in the particles of the invention in an amount from about 0 weight percent to about 60 weight percent. Preferably, the amino acid salt is present in the particles in an amount of about 5 to about 50 weight percent, about 5 to about 40 weight percent, about 5 weight percent to about 30 weight percent, for example, the amino acid can be present in an amount of about 20 to about 40 weight percent, about 25 to about 35 weight percent, or about 30 weight percent. Methods of forming which include an amino acid are described in U.S. patent application Ser. No. 09/382,959, filed on Aug. 25, 1999, entitled “Use of Simple Amino Acids to Form Porous Particles During Spray Drying” and U.S. patent application Ser. No. 09/644,320, filed on Aug. 23, 2000, entitled “Use of Simple Amino Acids to Form Porous Particles,” the teachings of both are incorporated herein by reference in their entirety.

The particles can further comprise a material having a carboxylate moiety such as one or more carboxylic acids, and salts thereof, which are distinct from the metal cation complexed biologically active agent. In one embodiment, the carboxylate moiety or carboxylic acid includes at least two carboxyl groups. Carboxylic acids include the salts thereof as well as combinations of two or more carboxylic acids and/or salts thereof. In a preferred embodiment, the carboxylic acid is a hydrophilic carboxylic acid or salt thereof. Suitable carboxylic acids include but are not limited to hydroxydicarboxylic acids (e.g., monohydroxydicarboxylic and dihydroxydicarboxylic acids), hydroxytricarboxilic acids (e.g., monohydroxytricarboxylic and dihydroxytricarboxylic acids), and the like. Citric acid and citrates, such as, for example sodium citrate, are preferred.

In one embodiment, the carboxylic acid and/or salt thereof can be present in the particles in an amount ranging from about 0 to about 80 weight percent such as less than about 70, 60, 50, or about 40 weight percent. In an another embodiment, the carboxylic acid and/or salt thereof can be present in the particles in an amount of about 5 to about 40, about 5 to about 30, or about 10 to about 20 weight percent.

It is understood that when the particles include a carboxylic acid, an amino acid, a surfactant or any combination thereof, interaction between these components of the particle and the multivalent metal cation component can occur. Such interactions can be used to facilitate the production of particles with the desired physical properties (i.e., thin walls, etc.) while maintaining the advantages with respect to improving the solubility and dissolution properties of the encapsulated drugs. In addition to improving solubility relative to the bulk drug (i.e., not in the instant formulation), the formulations can be optimized for a particular use to further improve solubilities and other desirable properties of the particulate composition.

The particles of the invention can be characterized by their matrix transition temperature. As used herein, the term “matrix transition temperature” refers to the temperature at which particles are transformed from glassy or rigid phase with less molecular mobility to a more amorphorus, rubbery or molten state or fluid-like phase. As used herein, “matrix transition temperature” is the temperature at which the structural integrity of a particle is diminished in a manner which imparts faster release of drug from the particle. Above the matrix transition temperature, the particle structure changes so that mobility of the drug molecules increases resulting in faster release. In contrast, below the matrix transition temperature, the mobility of the drug particles is limited, resulting in a slower release. The “matrix transition temperature” can relate to different phase transition temperatures, for example, melting temperature (Tₘ), crystallization temperature (T_{c}) and glass transition temperature (T_{g}) which represent changes of order and/or molecular mobility within solids. The term “matrix transition temperature,” as used herein, refers to the composite or main transition temperature of the particle matrix above which release of drug is faster than below.

Experimentally, matrix transition temperatures can be determined by methods known in the art, in particular by differential scanning calorimetry (DSC) or other calorimetric measurements. Other techniques to characterize the matrix transition behavior of particles or dry powders include synchrotron X-ray diffraction, freeze fracture electron microscopy, and hot stage microscopy.

Matrix transition temperatures can be employed to fabricate particles having desired drug release kinetics and to optimize particle formulations for a desired drug release rate. Particles having a specified matrix transition temperature can be prepared and tested for drug release properties by in vitro or in vivo release assays, pharmacokinetic studies and other techniques known in the art. Once a relationship between matrix transition temperatures and drug release rates is established, desired or targeted release rates can be obtained by forming and delivering particles which have the corresponding matrix transition temperature. Drug release rates can be modified or optimized by adjusting the matrix transition temperature of the particles being administered.

The particles of the invention include materials which promote or impart to the particles a matrix transition temperature that yields a desired or targeted drug release rate. Properties and examples of suitable materials are further described below.

Rapid release of a drug (e.g., more rapid than physiological uptake) is observed with materials, which, when combined, result in a low matrix transition temperatures. As used herein, “low transition temperature” refers to particles which have a matrix transition temperature which is below or about the physiological temperature of a subject.

As used herein, “physiological temperature” generally refers to the normal body temperature of a human subject, for instance about 37° C., or the body temperature of a veterinary subject.

Combining appropriate amount of materials to produce particles having a desired transition temperature can be determined experimentally, for example by forming particles having varying proportions of the desired materials, measuring the matrix transition temperatures of the mixtures (for example by DSC), selecting the combination having the desired matrix transition temperature and, optionally, further optimizing the proportions of the materials employed.

In one embodiment, the particles of the invention include a phospholipid. Alternatively, the particles include a combination of phospholipids. Two or more phospholipids can be employed. Phospholipids suitable for oral delivery to a human subject are preferred.

Examples of phospholipids include, but are not limited to, phosphatidic acids, phosphatidylcholines, phosphatidylethanolamines, phosphatidylglycerols, phosphatidylserines, phosphatidylinositols or a combination thereof. Modified phospholipids for example, phospholipids having their head group modified, e.g., alkylated or polyethylene glycol (PEG)-modified, also can be employed. One or more of the phospholipids present in the particles can be charged. Examples of charged phospholipids are described in U.S. patent application Ser. No. 09/752,106, entitled “Particles for Inhalation Having Sustained Release Properties,” filed on Dec. 29, 2000, and in U.S. patent application Ser. No. 09/752,109, entitled Particles for Inhalation Having Sustained Release Properties, filed on Dec. 29, 2000; the entire contents of both these applications are incorporated herein by reference.

The phospholipid or combination of phospholipids can be present in the particles in an amount of about 1 to about 99 weight percent, such as about 1 to about 90 or about 5 to about 80 weight percent. Preferably, they can be present in the particles in an amount of about 10 to about 80 weight percent. In other embodiments, one or more phospholipids are present in an amount of about 5 to about 70, about 5 to about 60, about 5 to about 50, about 10 to about 40, or about 10 to about 30 weight percent such as, for example, about 5 to about 25, about 5 to about 15, or about 10 weight percent.

Suitable methods of preparing and administering particles which include phospholipids, are described in U.S. Pat. No. 5,855,913, issued on Jan. 5, 1999 to Hanes, et al., and in U.S. Pat. No. 5,985,309, issued on Nov. 16, 1999 to Edwards, et al. The teachings of both are incorporated herein by reference in their entirety.

The phospholipids or combinations thereof can be selected to impart controlled release properties to the particles of the invention. The phase transition temperature of a specific phospholipid or a combination of phospholipids can be below, around, or above the physiological temperature of a patient. By selecting phospholipids or combinations of phospholipids according to their phase transition temperature, the particles can be tailored to have a desired or targeted matrix transition temperature and, subsequently, controlled release and/or dissolution properties, such as to permit optimal solubility of a drug in the G.I. tract.

Particles comprising phospholipids or combinations thereof are described in U.S. Provisional Patent Application No. 60/150,742 entitled “Modulation of Release From Dry Powder Formulations by Controlling Matrix Transition,” filed on Aug. 25, 1999; in U.S. patent application Ser. No. 09/792,869 entitled “Modulation of Release From Dry Powder Formulations”, filed on Feb. 23, 2001; and in International Patent Application No. PCT/US02/05629 entitled “Modulation of Release From Dry Powder Formulations,” filed on Feb. 22, 2002, under Attorney Docket No, 2685.1012-010 and published as WO 02/067902 on Sep. 6, 2002. The contents of these three applications are incorporated by reference in their entirety.

Combining the appropriate amounts of two or more phospholipids to form a combination having a desired phase transition temperature is described, for example, in the Phospholipid Handbook (Gregor Cevc, editor, Marcell-Dekker, Inc., 1993).

The amounts of phospholipids to be used to form particles having a desired or targeted matrix transition temperature can be determined experimentally, for example by forming mixtures in various proportions of the phospholipids of interest, measuring the transition temperature for each mixture, and selecting the mixture having the targeted transition temperature.

Phospholipids have characteristic phase transition temperatures, as defined by the melting temperature (Tₘ), the crystallization temperature (T_{c}) and the glass transition temperature (T_{g}). Tₘ, T_{c} and T_{g} are terms known in the art. For example, these terms are discussed in Phospholipid Handbook (Gregor Cevc, editor, Marcel-Dekker, Inc., 1993).

Phase transition temperatures for phospholipids or combinations thereof can be obtained from the literature. Sources listing phase transition temperature of phospholipids are, for instance, the Avanti® Polar Lipids (Alabaster, Ala.) Catalog or the Phospholipid Handbook (Gregor Cevc, editor, Marcel-Dekker, Inc., 1993) Small variations in transition temperature values listed from one source to another may be the result of experimental conditions such as moisture content or other measurement techniques.

Experimentally, phase transition temperatures can be determined by methods known in the art, in particular by differential scanning calorimetry or other calorimetric measurements. Other techniques to characterize the phase behavior of phospholipids or combinations thereof include synchrotron X-ray diffraction, freeze fracture electron microscopy, and hot stage microscopy.

Examples of phospholipids having transition temperatures which are less or about the physiological temperature of a patient, are listed in Table 1. These phospholipids are referred to herein as having low transition temperatures. The values of the transition temperatures shown in Tables 1 were obtained from the Avanti® Polar Lipids (Alabaster, Ala.) Catalog.

TABLE 1
Phospholipids suitable for use in instant invention

| | | Transition |
|---|---|---|
| | | Tempera- |
| | Phospholipids | ture |
| 1 | 1,2-Dilauroyl-sn-glycero-3-phosphocholine (DLPC) | −1° C. |
| 2 | 1,2-Ditridecanoyl-sn-glycero-3-phosphocholine | 14° C. |
| 3 | 1,2-Dimyristoyl-sn-glycero-3-phosphocholine (DMPC) | 23° C. |
| 4 | 1,2-Dipentadecanoyl-sn-glycero-3-phosphocholine | 33° C. |
| 5 | 1,2-Dipalmitoyl-sn-glycero-3-phosphocholine (DPPC) | 41° C. |
| 6 | 1-Myristoyl-2-palmitoyl-sn-glycero-3-phosphocholine | 35° C. |
| 7 | 1-Myristoyl-2-stearoyl-sn-glycero-3-phosphocholine | 40° C. |
| 8 | 1-Palmitoyl-2-myristoyl-sn-glycero-3-phosphocholine | 27° C. |
| 9 | 1-Stearoyl-2-myristoyl-sn-glycero-3-phosphocholine | 30° C. |
| 10 | 1,2-Dilauroyl-sn-glycero-3-phosphate (DLPA) | 31° C. |
| 11 | 1,2-Dimyristoyl-sn-glycero-3-[phospho-L-serine] | 35° C. |
| 12 | 1,2-Dimyristoyl-sn-glycero-3-[phospho-rac-(1-glycerol)] | 23° C. |
| | (DMPG) | |
| 13 | 1,2-Dipalmitoyl-sn-glycero-3-[phospho-rac-(1-glycerol)] | 41° C. |
| | (DPPG) | |
| 14 | 1,2-Dilauroyl-sn-glycero-3-phosphoethanolamine | 29° C. |
| | (DLPE) | |

In one embodiment, the particles of the invention include a combination of phospholipids. Two or more phospholipids can be present in the combination. In another embodiment, at least two of the phospholipids in the combination are miscible in one another.

Miscibilities of phospholipids are properties that are known in the art. As used herein, miscibility can be perfect, resulting in ideal mixing, and an absence of broadening of the phase transition in the mixture. As used herein, miscibility also can be high, resulting in mixing which is ideal or very nearly so, and a phase transition which is broader than the phase transitions of the pure components. As used herein, miscibility also can be moderate, which, upon mixing results in solidus curves in the phase diagram which are not flat over any significant range of compositions. Miscibilities of many phospholipids in binary mixtures are available in the literature, for example in the Avanti® Polar Lipids (Alabaster, Ala.) Catalog. See also Thermotropic Phase Transitions of Pure Lipids in Model Membranes and Their Modifications by Membrane Proteins, Dr. J. R. Silvus, Lipid-Protein Interactions, John Wiley & Sons, Inc., New York, 1982. Miscibilities of phospholipids also can be determined experimentally, as known in the art.

The effects of phospholipid miscibility on the matrix transition temperature of the phospholipid mixture can be determined by combining a first phospholipid with other phospholipids having varying miscibilities with the first phospholipid and measuring the transition temperature of the combinations.

Without wishing to be bound by any particular interpretation of the invention, it is believed that materials which are highly or perfectly miscible in one another tend to yield an intermediate overall matrix transition temperature, all other things being equal. On the other hand, materials which are immiscible in one another tend to yield an overall matrix transition temperature that is governed either predominantly by one component or may result in biphasic release properties.

Such combinations are described in U.S. Provisional Application No. 60/150,662, filed on Aug. 25, 1999, entitled “Formulation for Spray-Drying Large Porous Particles,” and U.S. Non-Provisional patent application Ser. No. 09/644,105 filed on Aug. 23, 2000, titled “Formulation for Spray-Drying Large Porous Particles”; the teachings of both are incorporated herein by reference in their entirety.

Preferred combinations of phospholipids include: 1,2-dipalmitoyl-sn-glycero-3-phosphocholine (DPPC) and-1,2-distearoyl-sn-glycero-3-phosphocholine (DSPC); 1,2-distearoyl-sn-glycero-3-phosphocholine (DSPC) and 1,2-dipalmitoyl-sn-glycero-3-[phospho-rac-(1-glycerol)] (DPPG); and 1,2-dipalmitoyl-sn-glycero-3-phosphocholine (DPPC) and 1,2-distearoyl-sn-glycero-3-[phospho-rac-(1-glycerol)] (DSPG).

Suitable ratios of phospholipid amounts to be employed in forming the particles of the invention that result in the desired drug release kinetics can be determined experimentally.

The particles can include one or more additional materials. Optionally, at least one of the one or more additional materials also is selected in a manner such that its combination with the phospholipids discussed above results in particles having a matrix transition temperature which results in the targeted or desired drug release rate.

Other materials, such as materials which promote controlled release kinetics of the medicament can also be employed. For example, biocompatible, and preferably biodegradable polymers can be employed. Particles including such polymeric materials are described in U.S. Pat. No. 5,874,064, issued on Feb. 23, 1999 to Edwards, et al., the teachings of which are incorporated herein by reference in their entirety.

The particles of the invention have specific drug release properties. Drug release rates can be described in terms of the half-time of release of a bioactive agent from a formulation. As used herein the term “half-time” refers to the time required to release 50% of the initial drug payload contained in the particles. Fast drug release rates generally are less than 30 minutes and range from about 1 minute to about 60 minutes. Controlled release rates generally are longer than 2 hours and can range from about 1 hour to about several days.

Drug release rates can also be described in terms of release constants. The first order release constant can be expressed using one of the following equations:

M_{pw(t)}=M_{(∞)}*e^{-k*t} (1)

or,

M₍ₜ₎=M_{(∞)}*(1−e^{-k*t}) (2)

Where k is the first order release constant. M_{(∞)} is the total mass of drug in the drug delivery system, e.g. the dry powder, and M_{pw(t)} is drug mass remaining in the dry powders at time t. M₍ₜ₎ is the amount of drug mass released from dry powders at time t. The relationship can be expressed as:

M_{(∞)}=M_{pw(t)}+M₍ₜ₎ (3)

Equations (1), (2) and (3) may be expressed either in amount (i.e., mass) of drug released or concentration of drug released in a specified volume of release medium.

For example, Equation (2) may be expressed as:

C₍ₜ₎=C_{(∞)}*(1−e^{-k*t}) (4)

Where k is the first order release constant. C_{(∞)} is the maximum theoretical concentration of drug in the release medium, and C₍ₜ₎ is the concentration of drug being released from dry powders to the release medium at time t.

The “half-time” or t_{50%} for a first order release kinetics is given by a well-know equation,

t_{50%} =0.693/k (5)

Drug release rates in terms of first order release constant and t_{50%} may be calculated using the following equations:

k=−ln(M_{pw(t)}/M_{(∞)}/t (6)

or,

k=−ln(M_{(∞)}−M₍ₜ₎)/M_{(∞)}/t (7)

In one embodiment, the particles of the invention exhibit drug release rates that equal or exceed the rate of drug uptake at the site of delivery, such that the dissolution rate is no longer the limiting factor in establishing the pharmacokinetic/pharmacodynamic profile of the drug.

By determining a baseline of drug uptake, it is believed that adjusting particle characteristics as disclosed herein, favorably affects the pharmacokinetic/pharmacodynamic profile to achieve the desired result. Particle characteristics which can be varied include, but are not limited to, geometric diameter, aerodynamic diameter, tap density, mass density, wall thickness and morphology.

Applicants have discovered a particularly important feature of the invention, for conferring pharmacokinetic/pharmacodynamic relevant solubility to a poorly soluble drug. In one embodiment, the feature is dissolving a crystalline drug to form a solution and spray drying the solution thereby making the drug amorphous and small without damaging bioactivity and concurrently making the particle comprising the amorphous drug. The combination of now small and amorphous drug imbedded in the amorphous thin walled particle confers surprising solubility when compared to the bulk drug.

There is a relationship between the tap density and the mean wall thickness for particles. This constrains the maximum domain size of solid material and hence contributes to improved dissolution when the wall thickness in less than 1 micron

In one embodiment, the particles of the invention have a tap density less than about 0.4 g/cm³. Particles which have a tap density of less than about 0.4 g/cm³ are referred herein as “large porous particles.” More preferred are particles having a tap density less than about 0.3, less than 0.2, or less than about 0.1 g/cm³. Tap density can be determined using the method of USP Bulk Density and Tapped Density, United States Pharmacopeia convention, Rockville, Md., 10^{th} Supplement, 4950-4951, 1999. Instruments for measuring tap density, known to those skilled in the art, include but are not limited to the Dual Platform Microprocessor Controlled Tap Density Tester (Vankel, N.C.) or a GeoPyc™ instrument (Micrometrics Instrument Corp., Norcross, Ga. 30093). Tap density is a standard measure of the envelope mass density. The envelope mass density of an isotropic particle is defined as the mass of the particle divided by the minimum sphere envelope volume within which it can be enclosed. Features which can contribute to low tap density include irregular surface texture and porous structure.

The diameter of the particles, for example, their volumetric median geometric diameter (VMGD), can be measured using an electrical zone sensing instrument such as a Multisizer IIe, (Coulter Electronic, Luton, Beds, England), or a laser diffraction instrument such as HELOS (Sympatec, Princeton, N.J.). Other instruments for measuring particle geometric diameter are well known in the art. The diameter of particles in a sample will range depending upon factors such as particle composition and methods of synthesis. The distribution of size of particles in a sample can be selected to permit optimal solubility in the G.I. tract.

The particles of the present invention have a preferred size, e.g., a volumetric median geometric diameter (VMGD) of at least about 5 microns. In some embodiments, the VMGD of the particles is about 5 to about 50 microns, about 5 to about 30 microns such as, for example, the particles have a VMGD of about 5 to about 25 microns, about 5 to about 15 microns, or about 15 to about 30 microns. In other embodiments, the particles have a median diameter, mass median diameter (MMD), a mass median envelope diameter (MMED) or a mass median geometric diameter (MMGD) of at least about 5 microns, for example, about 5 to about 50 microns, about 5 to about 30 microns such as about 5 to about 25 microns, about 5 to about 15 microns, or about 15 to about 30 microns.

In one embodiment, the particles are characterized by their aerodynamic diameter and have an aerodynamic diameter such as, for example, a mass median aerodynamic diameter (MMAD) of about 1 to about 5 microns. Particles of the instant invention have a MAD of about 1 to about 3, about 2 to about 4, or about 3 to about 5 microns. Aerodynamic diameter can be measured using techniques known to those of skill in the art. For example, mass median aerodynamic diameter (MMAD) is determined using an API AeroDisperser, Model 3230, and Aerosizer, Model 3225 (TSI, Inc., St. Paul, Minn.).

Other suitable particles can be adapted for use in oral delivery as described herein, said particles being described in U.S. Provisional Patent Application No. 60/331,708, entitled “High Surface Area Particles for Inhalation,” filed on Nov. 20, 2001 under Attorney Docket No. 2685.2009-000; U.S. Provisional Patent Application No. 60/331,707, entitled “Compositions for Sustained Action Drug Delivery and Methods of Use Thereof,” filed on Nov. 20, 2001 under Attorney Docket No. 2685.2006-000; and U.S. Provisional Patent Application No. 60/356,660, entitled “Compositions for Sustained Action Drug Delivery and Methods of Use Thereof,” filed on Mar. 18, 2002. The contents of each of which are incorporated herein in their entirety by reference.

The dosage to be administered to the mammal, such as a human, will contain a therapeutically effective amount of a compound described herein. As used herein, the term “therapeutically effective amount” means the amount needed to achieve the desired therapeutic or diagnostic effect or efficacy. The actual effective amounts of drug can vary according to the biological activity of the particular compound employed; specific drug or combination thereof being utilized; the particular composition formulated; the mode of administration; the age, weight, and condition of the patient; the nature and severity of the symptoms or condition being treated; the frequency of treatment; the administration of other therapies; and the effect desired. Dosages for a particular patient can be determined by one of ordinary skill in the art using conventional considerations (e.g. by means of an appropriate, conventional pharmacological protocol).

For general information concerning formulations, see e.g., Gilman, et al. (eds.), 1990, Goodman and Gilman's: The Pharmacological Basis of Therapeutics, 8^{th} ed., Pergamon Press; and Remington's Pharmaceutical Sciences, 17^{th} ed., 1990, Mack Publishing Co., Easton, Pa.; Avis, et al. (eds.), 1993, Pharmaceutical Dosage Forms: Parenteral Medications, Dekker, New York; Lieberman, et al. (eds.), 1990, Pharmaceutical Dosage Forms: Disperse Systems, Dekker, New York.

The compounds of the present invention can be administered in conventional pharmaceutical administration forms, for example, uncoated or (film-)coated tablets, capsules, powders, granules, suppositories, suspensions or solutions. These are produced in a conventional manner. The active substances can for this purpose be processed with conventional pharmaceutical aids such as tablet binders, fillers, preservatives, tablet disintegrants, flow regulators, plasticizers, wetting agents, dispersants, emulsifiers, solvents, sustained release compositions, and/or antioxidants (cf. H. Sücker, et al.,: Pharmazeutische Technologie, Thieme-Verlag, Stuttgart, 1978). The administration forms obtained in this way typically contain from about 1 to about 90 percent by weight of the active substance.

In one embodiment, the particles are in the form of a powder and are enclosed or stored in a capsule. The capsule is filled with particles and/or compositions comprising particles, as known in the art. For example, vacuum filling or tamping technologies may be used. Generally, filling the capsule with powder can be carried out by methods known in the art. In one embodiment of the invention, the particles which is enclosed or stored in a capsule has a mass of at least about 1.5 mg. In a second embodiment, the mass of the particles stored or enclosed in the receptacle comprises a mass of bioactive agent from at least about 5 mg. In a third embodiment, the mass of the particles stored or enclosed in the receptacle comprises a mass of bioactive agent from at least about 250 mg milligrams. Capsules are designated with a particular capsule size, such as 2, 1, 0, 00 or 000. Suitable capsules can be obtained, for example, from Shionogi (Rockville, Md.).

In one embodiment, the particles of the invention have a dissolution rate enhancement of at least 2-fold compared to the bulk drug. In a second embodiment, the particles of the invention have a dissolution rate enhancement of at least 10-fold.

In a second embodiment, the particles of the invention have a dissolution rate enhancement of about 2-fold to about 10-fold compared to the bulk drug. In a more preferred embodiment, the particles have a dissolution rate enhancement of about 10-fold to about 25-fold compared to the bulk drug. Dissolution rate can be determined using the method of the USP Drug Product Dissolution Test United States Pharmacopeia convention, Rockville, Md. Instruments for measuring tap density, known to those skilled in the art, include but are not limited to the VanKel Model DT1™ rotating basket apparatus (VanKel Corp., Cary, N.C. 27513).

The particles can be fabricated with a rough surface texture to reduce particle agglomeration and improve flowability of the powder. The spray-dried particles have improved solubility properties. The spray-dried particles can be fabricated with features which enhance solubility. The spray-dried particles can be fabricated with features that increase surface area and decrease diffusion distance which enhance dissolution.

In a preferred embodiment, suitable particles are fabricated by spray drying. In one embodiment, the method includes forming a mixture including one or more highly insoluble agents such as, but not limited to, danazol, glyburide, glipizide, piroxicam, lansoprazole, ketoprofen, cortisone, cyclosporine, dihydrotachysterol, dipyridamole, dronabinol, ergotamine, ethinyl estradiol, felodipine, finasteride, fluphenazine, griseofulvin, isotretinoin, loratidine, polythiazide, reserpine, tacrolimus, altretamine, triazolam, astemizole, carvedilol, digoxin, estradiol, glimepiride, hydrochlorothiazide, indapamide, isomethetene, letrozole, leucovorin, folinic acid, leukeran, melphalan, nifepidine, nimopidine, nisoldipine, oxazepam, perphenazine, simvastatin, spironolactone, zafirlukast, estazolam and olanzapine, or a combination thereof, and one or more surfactants, such as, for example, one or more of the surfactants described herein. In a preferred embodiment, the mixture includes one or more phospholipids, such as, for example, one or more of the phospholipids described herein. The mixture employed in spray drying can include an organic or aqueous-organic solvent.

Suitable organic solvents that can be employed include, but are not limited to, alcohols, for example, ethanol, methanol, propanol, isopropanol, butanols, and others. Other organic solvents include, but are not limited, to perfluorocarbons, dichloromethane, chloroform, ether, ethyl acetate, methyl tert-butyl ether and others.

The total amount of solvent or solvents being employed in the mixture being spray dried generally is greater than about 99 percent by weight. The amount of solids (drug, charged lipid and other ingredients) present in the mixture being spray dried generally is less than about 1.0 weight percent. Preferably, the amount of solids in the mixture being spray dried ranges from about 0.05 to about 0.5 percent by weight.

In some embodiments, co-solvent systems are employed to spray dry particles. Co-solvents include an aqueous solvent and an organic solvent, such as, but not limited to, the organic solvents as described above. Aqueous solvents include water and buffered solutions. In one embodiment, an ethanol/water solvent is preferred with the ethanol/water ratio ranging from about 30:70 to about 90:10 ethanol:water.

The spray drying mixture can have a neutral, acidic or alkaline pH. Optionally, a pH buffer can be added to the solvent or co-solvent or to the formed mixture. Preferably, the pH can range from about 3 to about 10.

Suitable spray-drying techniques are described, for example, by K. Masters in “Spray Drying Handbook,” John Wiley & Sons, New York, 1984. Generally, during spray-drying, heat from a hot gas such as heated air or nitrogen is used to evaporate the solvent from droplets formed by atomizing a continuous liquid feed. Other spray-drying techniques are well known to those skilled in the art. In a preferred embodiment, a rotary atomizer is employed. Examples of suitable spray dryers using rotary atomization include the Mobile Minor Spray Dryer, manufactured by Niro, Denmark. The hot gas can be, for example, air, nitrogen or argon.

The spin-rate of the rotary atomizer can range from about 2,000 to about 55,000 rpm. The rotary atomizer may have from about 4 to about 24 vanes. The inlet temperature can range from about 80° C. to about 400° C. The outlet temperature can range from about 50° C. to about 130° C. The liquid feed rate can range from about 20 mL/min to about 120 mL/min. The gas feed rate can range from about 60 kg/h to about 120 kg/h.

Other particles, methods for production of particles, and methods of administering particles are described in U.S. patent application Ser. No. 09/878,146, filed on Jun. 8, 2001, entitled “Method and Apparatus for Producing Dry Highly Efficient Delivery Of A Large Therapeutic Mass Aerosol;” U.S. patent application Ser. No. 09/837,620, filed on Apr. 18, 2001, entitled “Control Of Process Humidity To Produce Large, Porous Particles;” International Patent Application No. PCT/US02/12320 entitled “Control Of Process Humidity To Produce Large, Porous Particles,” filed on Apr. 17, 2002, and published as WO 02/085326 on Oct. 31, 2002; U.S. patent application Ser. No. ______, entitled “Improved Particulate Compositions for Pulmonary Delivery,” filed on even date herewith under Attorney Docket No. 2685.2009-001; and U.S. patent application Ser. No. ______, entitled “Compositions for Sustained Action Product Delivery and Methods of Use Thereof,” filed on even date herewith under Attorney Docket No. 2685.2006-002. Methods and apparatus for producing dry particles are discussed in U.S. patent application Ser. No. 10/101,563, entitled “Method and Apparatus for Producing Dry Particles,” filed on Mar. 20, 2002. The entirety of each of these applications is incorporated herein by reference.

The term, “Poorly soluble,” is commonly used and refers herein to a solubility of <10-100 mg/L in a “physiologically-relevant medium.”“Physiologically-relevant medium” is a solution that simulates G.I. conditions and can vary based on the target area of the dosage form. Examples of suitable physiologically-relevant media include phosphate buffered saline (PBS, pH 7.4), simulated gastric fluid without enzymes (SGF), simulated intestinal fluid without enzymes (SIF), aqueous media with synthetic surfactants (e.g., sodium lauryl sulfate), and acidic solutions (0.1 N HCl).

“Rapid dissolution” is generally considered relative an unimproved raw material (such as bulk crystalline drug). An improvement in dissolution is usually considered on the order of about 2- to about 10-fold.

“Dissolution” refers to a change in a change of state, from a solid state to a dissolved state (i.e., solvated in a water-rich environment such as gastrointestinal tract fluid.

“Dissolution rate” is fundamental physical property measuring mass per area multiplied by time so it is always in flux. Units for dissolution can be absolute (i.e., per area) including mg/min/meter or relative (i.e., no area term) including mg/min.

“Flowability” refers to a powder characteristic that affects the ease of processing. For a material to be considered to be suitably flowable, it must be amenable to processing in automated equipment (such as capsule fillers or tablet making machines) using industry standard techniques. Flowability is measured using a number of techniques referred to as powder rheometric methods such as shear cell methods and dynamic angles of repose.

“Wettability” is a property that affects the interaction of the powder in water. Wettability is a function of surface properties such as surface energy (surface tension) and morphology. This property can be measured using instruments such as dynamic vapor sorption or BET analyzers. Suitable units include water percent weight gain.

The term “oral” means taken by mouth.

“Alimentary canal” refers to the tubular passage that extends from the mouth to anus and functions in digestion and absorption of food and elimination of residual waste.

“Disintegration” refers to the breakdown of the particle wall.

“Degradation” refers to the breakdown of chemical structure for the active drug substance.

“Diffusion” refers to the tendency of molecules to migrate from an area of high concentration to an area of low concentration.

“Diffusion distance” refers to the distance a molecule must travel to exit the particle wall and reach the solution that it is dissolving in.

“Maximum diffusion distance” represents ½ the wall thickness.

The term “bulk drug” refers to the pure form of a drug resulting from the drug chemical manufacturing process. The drug can be present in the form of a salt. The bulk drug is usually a highly refined crystalline form of the drug suitable for compounding with excipients and forming tablets or filling capsules. Bulk drug particles typically range in size from about 5 microns to greater than about 100 microns.

The invention is illustrated by the following examples which are not intended to be limiting in any way.

### EXEMPLIFICATION

### Example 1

Many potential candidates were screened during this process. An original candidate list was compiled by searching the Physicians' Desk Reference, PDR, 55^{ed} Medical Economics Co., Montvale, N.J. (2001), for all low dose, poorly soluble, FDA approved drugs. This list was then prioritized based on aqueous solubility, safety, and cost. The first criteria the compound had to pass was that they are poorly soluble in aqueous media (phosphate buffered saline, PBS pH 7.4 was used). This test was done by mixing 0.3-1.0 L of PBS and adding 10 mg of bulk drug. The drug needed to be no more soluble than 20 mg/L in PBS pH 7.4 to be considered for further testing. If the drug fully dissolved at this concentration, it was not considered for the testing. Ketoprofen is the only exception to this, but it is poorly soluble in acidic conditions and met this criteria when tested in phosphoric acid solution of pH 3.

### Example 2

The compounds that passed the initial screening: danazol, glyburide, glipizide, piroxicam, lansoprazole, and ketoprofen; were then individually spray dried into formulations of the instant invention. The particles were spray dried in a Niro Mobile-Minor Spray Dryer, size 0 (Niro, Inc., Denmark). The solution was fed into the dryer at 70 mL/min and atomized using a V24 atomizer at 22,000 RPM.

For each experiment, the powder used for the control was made by mixing bulk drug and excipients in the same proportion as the spray dried instant formulation.

### Example 3

Analytical Methods

Dissolution Testing

The dissolution testing was performed in a VanKel Model DT1™ rotating basket apparatus. Powder was placed into a basket that was made of a 50 micron mesh. This basket was then immersed into 500 mL of dissolution media and rotated at 200 RPM. The media was pH 7.4 phosphate buffered saline for the piroxicam, lansoprazole, glipizide and glyburide. The media for the danazol was PBS with 0.05% sodium dodecyl sulfate and for the ketoprofen it was phosphate buffered saline adjusted to pH 3.0 with phosphoric acid. Samples were taken at 1, 2, 3, 5, 10 and 15 minutes and analyzed.

Sample Preparation

2 mL of sample was taken using a disposable syringe and filtered through a 0.45 micron filter to remove any remaining particulates. The sample was then placed into a quartz cuvet or HPLC sample vials depending on the associated analytical method. The danazol samples needed further adjustments before they could be analyzed. The surfactant present in the dissolution media stuck onto the HPLC column and caused results to vary dramatically. The 2 mL sample was placed into a vial and 500 mg of NaCl were added. 2 mL of hexane were added to this solution and the vial was shaken for 30 seconds. 0.5 mL of the hexane layer was then removed and placed into an HPLC vial for analysis.

Dissolution Profiles

The cumulative amount of drug dissolved was expressed as a percentage of the initial total drug deposited and plotted against time. Dissolution profiles were fitted to the first order release equation:

C₍ₜ₎=C_{(inf)}*(1−e^{-k*t})

where, k is the first order release constant, C₍ₜ₎ is the concentration of drug at time t (min) and C_{(inf)} is the maximal theoretical drug concentration in the dissolution medium.

### Example 4

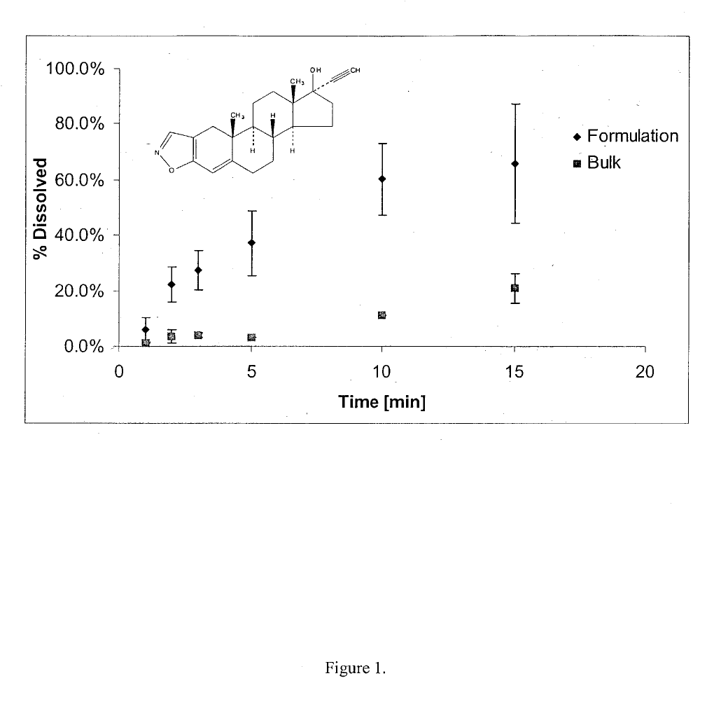

Danazol is an endocrine regulator with a therapeutic dose of 200 mg. Danazol is the least soluble compound of the group that was tested. The structure can be seen in FIG. 1. The danazol was spray dried in a Niro Mobile-Minor Spray Dryer, size 0. The solution was a 50/50-volume mixture of ethanol and water with 20/40/30/10-weight percentage danazol/maltodextrin/leucine/DPPC at a concentration of 1 g/L. 10 g/L of ammonium bicarbonate was added as a volatilizing agent, but was not present in the final product. The temperature at the inlet was 148° C. The solution was fed into the spray dryer at 70 mL/min and atomized using a V24 atomizer at 22000 RPM. This resulted in an outlet temperature of 60° C. The powder yield was 33% with a mass median aerodynamic diameter of 2.223 microns at 1 bar and a volumetric median geometric diameter of 11.81 microns at 1 bar.

The danazol samples were analyzed via reverse-phase HPLC using a Waters 2470 dual wavelength analyzer with a model 600 control system. The stationary phase was a Phenomonex C-18 column. The mobile phase was 1 mL/min of a 40/25/35 mixture of acetonitrile/water/methanol. The eluent was analyzed at a wavelength of 284 nanometers. The formulation that showed the largest relative gain in dissolution rate versus bulk drug was a 20/40/30/10 mixture of danazol/maltodextrin/leucine/DPPC. The results of the dissolution testing are shown in FIG. 1. The initial dissolution rate of the instant formulation was 0.91 mg/min. The initial dissolution rate of the bulk formulation was 0.13 mg/min. The initial dissolution rate of the instant formulation was 6.9 times that of the bulk material. The dissolution kinetics are well known and documented.

### Example 5

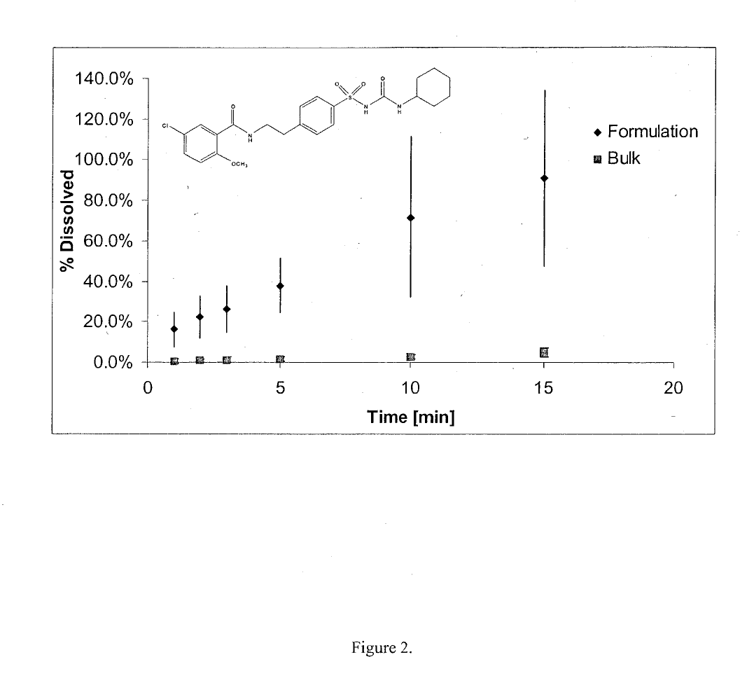

Glyburide is a sulfonylurea class compound that has an indication for glucose control. The therapeutic dose is 2.5 mg. Glyburide, also called glibenclamide, is known chemically as 1-[[p-[2-(5-chloro-o-anisamido)ethyl]phenyl]-sulfonyl]-3-cyclohexylurea and the molecular weight is 493.99. The structure of glyburide is shown in FIG. 2. The glyburide was spray dried in a Niro Mobile-Minor Spray Dryer, size 0. The solution was a 50/50-volume mixture of ethanol and water with 20/40/30/10-weight percentage glyburide/maltodextrin/leucine/DPPC at a concentration of 1 g/L. 10 g/L of ammonium bicarbonate was added as a volatilizing agent, but was not present in the final product. The temperature at the inlet was 148° C. The solution was fed into the spray dryer at 70 mL/min and atomized using a V24 atomizer at 22000 RPM. This resulted in an outlet temperature of 61° C. The powder yield was 33.5% with a mass median aerodynamic diameter of 2.201 microns and volumetric median geometric diameter of 11.50 microns at 1 bar.

The glyburide was analyzed via reverse-phase HPLC using a Waters 2470 dual wavelength analyzer with a model 600 control system. The stationary phase was a Phenomonex C-18 column while the mobile phase was 1.0 mL/min of 50/50 acetonitrile/aqueous buffer. The buffer was 0.05 mM Potassium Phosphate. The eluent was analyzed at a wavelength of 300 nanometers.

The results of the dissolution testing are shown in FIG. 2. The initial dissolution rate of the instant formulation was 0.87 mg/min. The initial dissolution rate of the bulk formulation was 0.04 mg/min. The initial dissolution rate of the instant formulation was over 21.7 times the rate of the bulk formulation. The dissolution kinetics are known to be enhanced by normal techniques and are well documented.

### Example 6

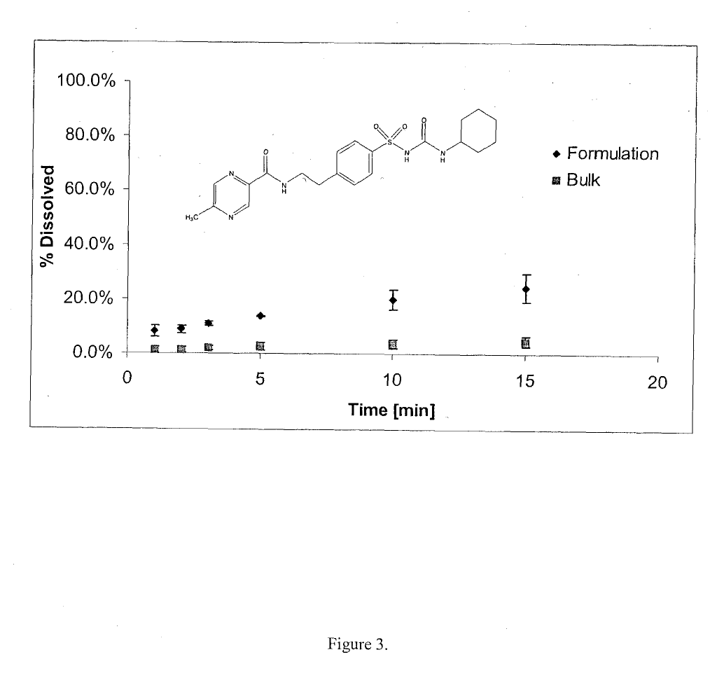

Glipizide, like its sister compound glyburide, is a sulfonylurea used as a glucose control agent, but the therapeutic dose is only 1 mg. The structure of glipizide is shown in FIG. 3. The glipizide was spray dried in a Niro Mobile-Minor Spray Dryer, size 0. The solution was a 50/50-volume mixture of ethanol and water with 30/40/30-weight percentage glipizide/maltodextrin/leucine at a concentration of 1 g/L. 10 g/L of ammonium bicarbonate was added as a volatilizing agent, but is not present in the final product. The temperature at the inlet was 150° C. The solution was fed into the spray dryer at 70 mL/min and atomized using a V24 atomizer at 22000 RPM. This resulted in an outlet temperature of 63° C. The powder yield was 13.8% with a mass median aerodynamic diameter of 2.975 microns and volumetric median geometric diameter of 11 microns at 1 bar.

The glipizide samples were analyzed using a Beckman DU 640 UV/VIS spectrophotometer at a wavelength of 272 nanometers.

The final dissolution kinetics can be seen in FIG. 3. The instant formulation had an initial dissolution rate of 0.38 mg/min. The bulk formulation had an initial dissolution rate of 0.07 mg/min. The instant formulation had an initial dissolution rate 5.4 times greater than the bulk powder with excipients.

### Example 7

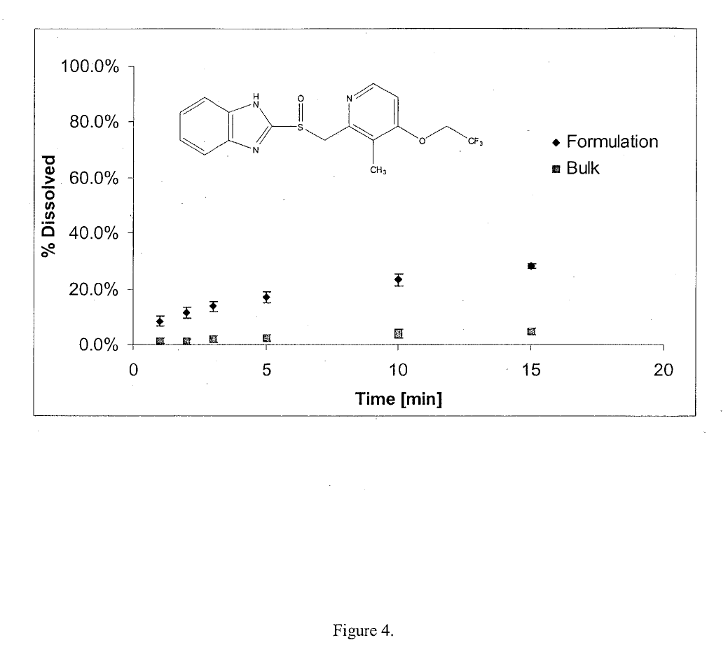

Lansoprazole is a proton pump inhibitor with a therapeutic dose of 15 mg. The structure of lansoprazole is shown in FIG. 4 The lansoprazole was spray dried in a Niro Mobile-Minor Spray Dryer, size 0. The solution was a 50/50-volume mixture of ethanol and water with 30/40/30-weight percentage lansoprazole/maltodextrin/leucine at a concentration of 1 g/L. 10 g/L of ammonium bicarbonate was added as a volatilizing agent, but was not present in the final product. The temperature at the inlet was 145° C. The solution was fed into the spray dryer at 70 mL/min and atomized using a V24 atomizer at 22000 RPM. This resulted in an outlet temperature of 65° C. The powder yield was 13.5% with a mass median aerodynamic diameter of 3.1 microns and a volumetric median geometric diameter of 10.98 microns at 1 bar.

The lansoprazole samples were analyzed using a Beckman DU 640 UV/VIS spectrophotometer at a wavelength of 280 nanometers.

Lansoprazole showed the one of the greatest gains in dissolution rate with the instant invention (see FIG. 4). The instant formulation had an initial dissolution rate that was 0.86 mg/min. The bulk formulation had an initial dissolution rate that was 0.07 mg/min. The instant formulation had an initial dissolution rate that was 13.1 times that of the bulk material. While lansoprazole is a poorly soluble compound, the bioavailability is not dissolution rate limited in the gut. This makes it an unfavorable drug for animal studies, but good as a proof of concept.

### Example 8

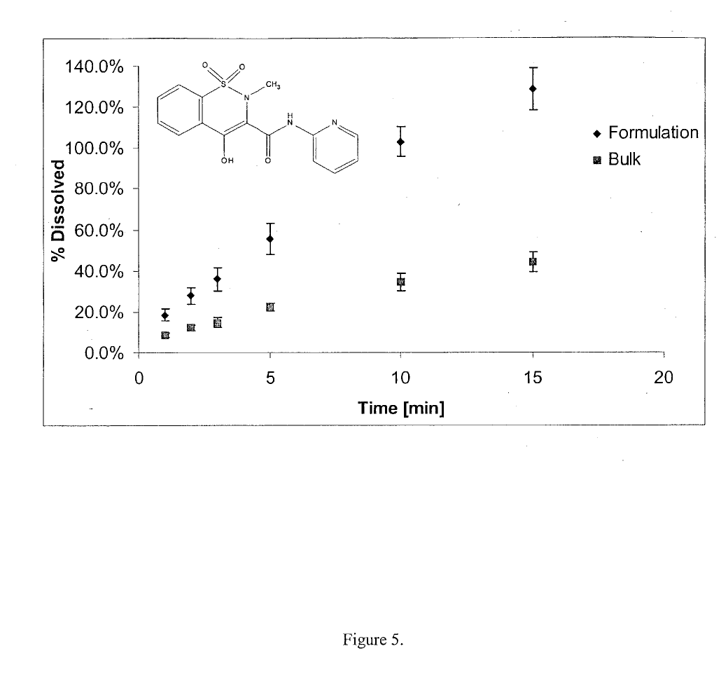

Piroxicam is COX1/COX2 inhibitor primarily used for treatment of arthritis. The therapeutic dose is 10 mg. The structure of piroxicam is shown in FIG. 5. The piroxicam was spray dried in a Niro Mobile-Minor Spray Dryer, size 0. The solution was a 50/50-volume mixture of ethanol and water with 30/40/30-weight percentage piroxicam/maltodextrin/leucine at a concentration of 1 g/L. 10 g/L of ammonium bicarbonate was added as a volatilizing agent, but is not present in the final powder. The temperature at the inlet was 155° C. The solution was fed into the spray dryer at 70 mL/min and atomized using a V24 atomizer at 22000 RPM. This resulted in an outlet temperature of 64° C. The powder had a mass median aerodynamic diameter of 2.84 microns and a volumetric median geometric diameter of 21.3 microns at 1 bar.

The piroxicam samples were analyzed using a Beckman DU 640 UV/VIS spectrophotometer at a wavelength of 260 nanometers.

The dissolution kinetics of piroxicam can be seen in FIG. 5. The instant formulation had an initial dissolution rate that was 0.90 mg/min. The bulk formulation had an initial dissolution rate that was 0.27 mg/min. The instant formulation had an initial dissolution rate that was 3.4 times that of the bulk material. The piroxicam powder degraded quickly, which may have an effect on the dissolution rate.

### Example 9

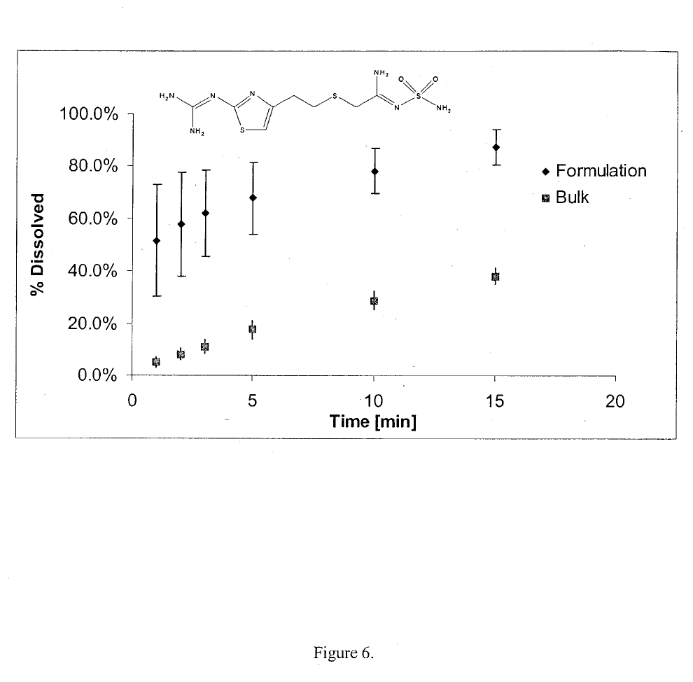

Ketoprofen is a COX1/COX2 inhibitor used for pain relief and has a therapeutic dose of 50 mg. The structure of ketoprofen is shown in FIG. 6. The ketoprofen was spray dried in a Niro Mobile Minor Spray Dryer, size 0. The solution was a 50/50-volume mixture of ethanol and water with 30/40/30-weight percentage ketoprofen/maltodextrin/leucine at a concentration of 1 g/L. 10 g/L of ammonium bicarbonate was added as a volatilizing agent, but was not present in the final product. The temperature at the inlet was 165° C. The solution was fed into the spray dryer at 70 mL/min and atomized using a V24 atomizer at 22000 RPM. This resulted in an outlet temperature of 62° C. The resulting powder had a mass median aerodynamic diameter of 4.12 microns and a volumetric median geometric diameter of 15 microns at 1 bar.

The ketoprofen samples were analyzed using a Beckman DU 640 UV/VIS spectrophotometer at a wavelength of 255 nanometers.

The dissolution kinetics of ketoprofen is shown in FIG. 6. The instant formulation had an initial dissolution rate that was 1.55 mg/min. The bulk formulation had an initial dissolution rate that was 0.54 mg/min. The instant formulation had an initial dissolution rate that was 2.8 times faster than the bulk powder. The dissolution rate enhancement was lower for ketoprofen than the other examples because bulk ketoprofen is more soluble than the bulk drugs of the other examples, even at very low pH. A greater solubility of the bulk drug means that the relative increase in dissolution rate is lower for ketoprofen than the drugs of the other examples.

### Example 10

This example describes additional dissolution kinetics testing of ketoprofen preformed using the powder production and testing procedures of Examples 9 and 3, respectively. The spray dried ketoprofen-containing powder had a volumetric median geometric diameter of 6.7 microns.

The instant formulation had an initial dissolution rate in dissolution medium at a pH of 7.4 that was 9.2 mg/min. The bulk formulation at that pH had an initial dissolution rate that was 4.4 mg/min. Therefore, the instant formulation had an initial dissolution rate that was 2.1 times faster than the bulk powder when tested at pH 7.4.

Dissolution testing was also preformed wherein the pH of the dissolution medium was 3.0. The instant formulation had an initial dissolution rate that was 2.4 mg/min. The bulk formulation at that pH had an initial dissolution rate that was 0.3 mg/min. Therefore, the instant formulation had an initial dissolution rate that was 8.1 times faster than the bulk powder when tested at pH 3.0.

### Example 11

This example describes a study conducted to determine the pharmacokinetic (PK) profile of an orally administered, dry powder formulation of glyburide. The experiment used male, Sprague-Dawley rats obtained from Taconic Farms (Germantown, N.Y.).

Dry powder particles containing 20% glyburide, 40% maltodextrin, 30% 1-leucine, and 10% DPPC were spray dried from a 50/50 (v/v) mixture of ethanol and water along with 10 g/L of ammonium bicarbonate using a method similar to that described in Example 5. The resulting powder had a mass median aerodynamic diameter of 2.3 microns and a volumetric median geometric diameter of 10 microns.

Three rats were selected from the general animal population. The average body weight of these animals was 372±9 grams (±SEM). Approximately 18-24 hours prior to dosing, rats were fitted with indwelling jugular catheters. Approximately 1.5 mg of dry powder particles containing 300 micrograms of glyburide was weighed into PCcaps™ (Capsugel, Greenwood, S.C.). Each rat was administered two glyburide containing capsules using an oral dosing tube.

Blood samples (0.3 mL) were collected at 0, 0.5, 1, 2 and 4 hours, where time 0 was the time of oral dosing. Blood was placed in microfuge tubes containing EDTA, and maintained on ice prior to centrifugation to obtain plasma. Plasma samples were stored at −20° C. until assayed for glyburide. Based on these data, it appeared that formulation of glyburide in large porous particles allowed for a more rapid uptake of the drug following oral administration that had been previously reported for traditional formulations of glyburide.

### Example 12

This example describes a study conducted to compare the pharmacokinetic (PK) profile of various orally administered formulations of glyburide. The experiment used male, Sprague-Dawley rats obtained from Taconic Farms (Germantown, N.Y.).

Dry powder particles containing 20% glyburide, 40% maltodextrin, 30% 1-leucine, and 10% DPPC were spray dried from a 50/50 (v/v) mixture of ethanol and water along with 10 g/L of ammonium bicarbonate using a method similar to that described in Example 5. The resulting powder had a mass median aerodynamic diameter of 2.3 microns and a volumetric median geometric diameter of 10 microns.

Rats were randomly assigned (average body weight 536±10 grams) to one of the following groups (n=6 rats per group):

a) Spray dried glyburide-containing powder (also referred to herein as ‘spray dried glyburide,’ prepared as above);

b) a mixture of glyburide and excipients used to used to produce spray dried glyburide, not formulated into large porous particles (also referred to herein as ‘bulk glyburide’);

c) an oral tablet formulation of micronized glyburide (Glynase; Pharmacia/Upjohn; also referred to herein as ‘micronized glyburide’); and

d) an oral tablet formulation of non-micronized glyburide (Micronase; Pharmacia/Upjohn; also referred to herein as ‘standard glyburide’).

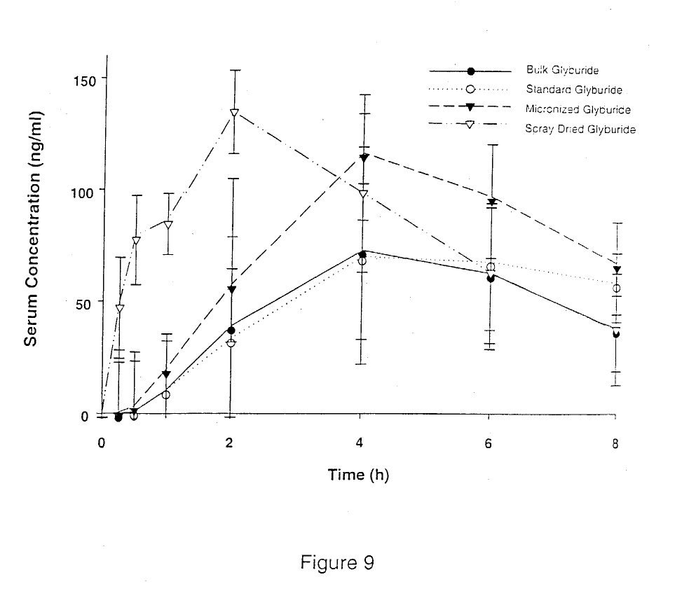

Each animal was administered a normal dose of 600 mg of glyburide. Due to the low density of the spray dried glyburide, the total amount of powder was distributed equally between 2 PCcap™ capsules (1.5 mg per capsule). For the other treatment groups, the dose of glyburide was administered using a single PCcap™ per animal. Approximately 18-24 hours prior to dosing rats were fitted with indwelling jugular catheters. Each rat was then administered glyburide containing capsules using an oral dosing tube. Blood samples (0.3 mL) were collected at 0, 0.5, 1, 2 and 4 hours, where time 0 was the time of oral dosing. Blood was placed in microfuge tubes containing EDTA, and maintained on ice prior to centrifugation to obtain plasma. Plasma samples were stored at −20° C. until assayed for glyburide. Rat blood serum concentration of glyburide (in nanograms per milliliter) versus time (in hours) is shown in FIG. 9. Area under the curve was determined for the time period up to 8 hours (AUC_{8 hours}) and is shown in Table 2.

TABLE 2
Area under the Curve up to 8 hours

| Formulation | AUC_{8 hours} |
|---|---|
| (a) Spray dried glyburide | 681 |
| (b) Bulk glyburide | 374 |
| (c) Micronized glyburide | 595 |
| (d) Standard glyburide | 390 |

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition for oral drug delivery comprising:
amorphous, hollow particles comprising regions of a poorly soluble agent embedded within the walls of said particles wherein the dissolution rate enhancement of the particles is between about 2-fold and about 25-fold compared to the agent in bulk form.

2. The composition of claim 1 wherein the poorly soluble agent is bioactive.

3. The composition of claim 2 wherein the bioactive agent is selected from the group consisting of small molecules, proteins, polypeptides and peptides.

4. The composition of claim 2 wherein the bioactive agent is selected from the group consisting of danazol, glyburide, glipizide, piroxicam, lansoprazole, ketoprofen, cortisone, cyclosporine, dihydrotachysterol, dipyridamole, dronabinol, ergotamine, ethinyl estradiol, felodipine, finasteride, fluphenazine, griseofulvin, isotretinoin, loratidine, polythiazide, reserpine, tacrolimus, altretamine, triazolam, astemizole, carvedilol, digoxin, estradiol, glimepiride, hydrochlorothiazide, indapamide, isomethetene, letrozole, leucovorin, folinic acid, leukeran, melphalan, nifepidine, nimopidine, nisoldipine, oxazepam, perphenazine, simvastatin, spironolactone, zafirlukast, estazolam and olanzapine.

5. The composition of claim 2 wherein the bioactive agent is selected from the group consisting of danazol, glyburide, glipizide, piroxicam, lansoprazole, and ketoprofen.

6. The composition of claim 1 wherein the poorly soluble agent has a solubility of less than about 100 mg/L.

7. The composition of claim 1 wherein the poorly soluble agent has a solubility of less than about 10 mg/L.

8. The composition of claim 1 wherein the particles have a dissolution rate enhancement of about 3-fold to about 10-fold compared to the agent in bulk form.

9. The composition of claim 1 wherein the particles have a dissolution rate enhancement of about 3-fold to about 5-fold compared to the agent in bulk form.

10. The composition of claim 1 wherein the walls of the particles have a wall thickness of less than about 1 micron.

11. The composition of claim 1 wherein the walls of the particles have a wall thickness of about 50 to about 400 nanometers.

12. The composition of claim 1 wherein the walls of the particles have a wall thickness of about 50 to about 200 nanometers.

13. The composition of claim 1 wherein the walls of the particles have a wall thickness of about 50 to about 100 nanometers.

14. The composition of claim 1 wherein the thicknesses of the regions of drug in the particle are about 20 nanometers to about 500 nanometers.

15. The composition of claim 1 wherein the thicknesses of the regions of drug in the particle are about 50 nanometers to about 400 nanometers.

16. The composition of claim 1 wherein the thicknesses of the regions of drug in the particle are about 100 nanometers to about 400 nanometers.

17. The composition of claim 1 wherein the thicknesses of the regions of drug in the particle are about 200 nanometers to about 400 nanometers.

18. The composition of claim 1 wherein the particles have a tap density of less than about 0.4 g/cm³.

19. The composition of claim 1 wherein the particles have a tap density of less than about 0.1 g/cm³.

20. The composition of claim 1 wherein at least 50% of the particles have a mean geometric diameter of about 5 microns to about 50 microns.

21. The composition of claim 1 wherein at least 50% of the particles have a mean geometric diameter of about 5 microns to about 15 microns and a tap density of less than about 0.1 g/cm³.

22. The composition of claim 1 further comprising a pharmaceutically acceptable carrier for oral administration.

23. The composition of claim 1 further comprising at least one excipient.

24. The composition of claim 23 wherein the excipient is selected from the group consisting of buffer salts, dextran, polysaccharides, lactose, trehalose, cyclodextrins, proteins, polycationic complexing agents, peptides, polypeptides, fatty acids, fatty acid esters, inorganic compounds, phosphates, lipids, sphingolipids, cholesterol, surfactants, polyaminoacids, polysaccharides, proteins, salts, gelatins, biodegradable polymers, and polyvinylpyrridolone.

25. The composition of claim 23 wherein the excipient is a biodegradable polymer.

26. The composition of claim 23 wherein the excipient is a polyester.

27. The composition of claim 23 wherein the excipient is a phospholipid.

28. A pharmaceutical composition for oral drug delivery comprising:
biodegradable particles comprising regions of a poorly soluble amorphous agent embedded within the walls of the particles wherein the average wall thickness is about 50 nanometers to about 500 nanometers, wherein the dissolution rate enhancement of the particles is about 2-fold to about 25-fold compared to the agent in bulk form.

29. A pharmaceutical composition for oral drug delivery comprising:
amorphous biodegradable particles comprising a poorly soluble therapeutic, prophylactic or diagnostic agent, wherein the particles have a tap density less than 0.4 g/cm³ and a mean geometric diameter of about 5 microns to about 30 microns which when administered orally have a dissolution rate enhancement of the particles of about 2-fold to about 25-fold compared to the agent in bulk form.

30. A method of treating a patient in need of a poorly soluble agent comprising:
orally administering to said patient an effective amount of amorphous, hollow particles comprising regions of a poorly soluble agent embedded within the walls of said particles wherein the dissolution rate enhancement of the particles is about 2-fold to about 25-fold compared to the agent in bulk form.

31. The method of claim 30 wherein the poorly soluble agent is bioactive.

32. The method of claim 31 wherein the bioactive agent is selected from the group consisting of small molecules, proteins, polypeptides and peptides.

33. The method of claim 31 wherein the agent is selected from the group consisting of danazol, glyburide, glipizide, piroxicam, lansoprazole, ketoprofen, cortisone, cyclosporine, dihydrotachysterol, dipyridamole, dronabinol, ergotamine, ethinyl estradiol, felodipine, finasteride, fluphenazine, griseofulvin, isotretinoin, loratidine, polythiazide, reserpine, tacrolimus, altretamine, triazolam, astemizole, carvedilol, digoxin, estradiol, glimepiride, hydrochlorothiazide, indapamide, isomethetene, letrozole, leucovorin, folinic acid, leukeran, melphalan, nifepidine, nimopidine, nisoldipine, oxazepam, perphenazine, simvastatin, spironolactone, zafirlukast, estazolam and olanzapine.

34. The method of claim 31 wherein the agent is selected from the group consisting of danazol, glyburide, glipizide, piroxicam, lansoprazole, and ketoprofen.

35. The method of claim 30 wherein the poorly water soluble agent has a solubility of less than about 100 mg/L.

36. The method of claim 30 wherein the poorly water soluble agent has a solubility of less than about 10 mg/L.

37. The method of claim 30 wherein the particles have a dissolution rate enhancement of about 2-fold to about 10-fold compared to the agent in bulk form.

38. The method of claim 30 wherein the particles have a dissolution rate enhancement of about 2-fold to about 5-fold compared to the agent in bulk form.

39. The method of claim 30 wherein the walls of the particles have a wall thickness of less than about 1 micron.

40. The method of claim 30 wherein the walls of the particles have a wall thickness of about 50 nanometers to about 400 nanometers.

41. The method of claim 30 wherein the walls of the particles have a wall thickness of about 50 to about 200 nanometers.

42. The method of claim 30 wherein the walls of the particles have a wall thickness of about 50 to about 100 nanometers.

43. The method of claim 30 wherein the thicknesses of the regions of drug in the particle are about 20 nanometers to about 500 nanometers.

44. The method of claim 30 wherein the thicknesses of the regions of drug in the particle are about 50 nanometers to about 400 nanometers.

45. The method of claim 30 wherein the thicknesses of the regions of drug in the particle are about 100 nanometers to about 400 nanometers.

46. The method of claim 30 wherein the thicknesses of the regions of drug in the particle are about 200 nanometers to about 400 nanometers.

47. The method of claim 30 wherein the particles have a tap density of less than about 0.4 g/cm³.

48. The method of claim 30 wherein the particles have a tap density of less than about 0.1 g/cm³.

49. The method of claim 32 wherein at least 50% of the particles have a mean geometric diameter of about 5 microns to about 50 microns.

50. The method of claim 30 wherein at least 50% of the particles have a mean geometric diameter of about 5 microns to about 15 microns and a tap density of less than about 0.1 g/cm³.

51. The method of claim 30 further comprising a pharmaceutically acceptable for oral administration.

52. The method of claim 30 further comprising at least one excipient.

53. The method of claim 52 wherein the excipient is selected from the group consisting of buffer salts, dextran, polysaccharides, lactose, trehalose, cyclodextrins, proteins, polycationic complexing agents, peptides, polypeptides, fatty acids, fatty acid esters, inorganic compounds, phosphates, lipids, sphingolipids, cholesterol, surfactants, polyaminoacids, polysaccharides, proteins, salts, gelatins, biodegradable polymers, and polyvinylpyrridolone.

54. The method of claim 52 wherein the excipient is a biodegradable polymer.

55. The method of claim 52 wherein the excipient is a polyester.

56. The method of claim 52 wherein the excipient is a phospholipid.

57. A method of administering to a patient in need of a poorly soluble agent comprising:
orally administering to said patient an effective amount of a particulate composition comprising amorphous, hollow particles comprising regions of a poorly soluble agent embedded within the walls of said particles wherein the dissolution rate enhancement of the particles is about 2-fold to about 25-fold compared to the agent in bulk form.

58. The method of claim 57 wherein the particulate composition is available in a capsule for oral drug delivery.

59. The method of claim 57 wherein the particulate composition is available in a tablet for oral drug delivery.

60. A process for making a particulate composition for oral drug delivery comprising spray drying a dilute solution comprising a poorly soluble agent;
wherein the particulate composition comprises amorphous, hollow particles comprising regions of a poorly soluble agent embedded within the walls of said particles wherein the dissolution rate enhancement of the particles is about 2-fold to about 25-fold compared to the agent in bulk form.

61. A particulate composition produced by the process of claim 60.

62. A pharmaceutical composition for oral drug delivery comprising:
amorphous, hollow particles comprising a poorly soluble agent molecularly dispersed within the walls of said particles wherein the dissolution rate enhancement of the particles is about 2-fold to about 25-fold compared to the agent in bulk form.

63. A pharmaceutical composition for oral drug delivery comprising:
amorphous, hollow particles comprising purely poorly soluble agent wherein the dissolution rate enhancement of the particles is about 2-fold to about 25-fold compared to the agent in bulk form.
